(19) United States
(12) Patent Application Publication
(10) Pub. No.: US 2019/0191957 A1
(43) Pub. Date: Jun. 27, 2019

(54) Method for having cleaner dishes

(71) Applicant: Vera Ann Flory, West Alexandria, OH (US)

(72) Inventor: Vera Ann Flory, West Alexandria, OH (US)

(73) Assignee: VA DESIGNS , Ltd., West Alexandria, OH (US)

(21) Appl. No.: 16/350,726

(22) Filed: Dec. 21, 2018

Related U.S. Application Data
(60) Provisional application No. 62/708,859, filed on Dec. 26, 2017.

(51) Int. Cl. A47L 15/00

(52) U.S. Cl. CPC A47L 15/0026; A47L 15/0047

(57) ABSTRACT

This method for cleaner dishes is a preferred method over prior art because it is ecology saver, saves money and energy resources while removing bacteria and generally making life more pleasant. This method uses a leak proof water tank with the dish washing machine to hold the water that is changed by Lightening Clean Technology™ and then is used in the last rinse. This makes the dishes genuinely clean as well as the quality of the environment. This invention detoxifies, deodorizes and disinfects dishes without using chemicals or too hot of water which can cause melted items and burns if unloaded too soon.

### CROSS REFERENCE TO RELATED APPLICATION

This patent application is related to a Provisional Patent Application filed on Dec. 23, 2017 and granted on Jan. 18, 2018 with country code and number of U.S. 62/708,859

### BACKGROUND OF THE INVENTION

### a—Field of invention

This invention generally relates to a method for having cleaner dishes.

### b—Background

People use smelly, toxic cleaners and other chemicals in their dishwashers and very hot water or steam which can melt plastic, or cause burns.

### BRIEF SUMMARY OF THE INVENTION

The object of this invention is to have cleaner washed dishes by detoxifying, deodorizing and disinfecting, without using harmful smelly chemicals and/or extremely hot water which can cause melting or burns if the dishwasher, pan-washer, glass-washer, tray washer, utensil washer or whatever it is called is unloaded too soon. It will also deodorize the dishwasher so that it doesn't have to be cleaned. Another object of this invention is saving money, energy resources, and the quality of the environment, while removing bacteria and toxins and generally making life more pleasant.

### BRIEF DESCRIPTION OF THE VIEWS OF THE DRAWINGS

There are no drawings for this invention because there are many different manufacturers of dishwashers and many different types, shapes and sizes.

### DETAILED DESCRIPTION OF THE INVENTION

This method for cleaner washed dishes is a preferred method over prior art because it is ecology safer, whereby destroying mold, mildew, tobacco, onions, garlic and other offensive smells of which some are toxic. The water heat can be reduced and cause less burns and less melted items, and still have bacteria free dishes. The quality of the environment can be helped by using this invention in removing toxins, bacteria and bad odors and generally making life more pleasant.

This method to have cleaner dishes uses an apparatus, called Lightning Clean Technology™, with a washing machine that cleans dishes and other paraphanalia. Said apparatus detoxifies, deodorizes and disinfects, saving money from extra heated water (energy resources), as well as not having to replace ruined items.

A leak-proof container to hold water that the apparatus makes special, being put where it is most convenient for the type and shape of the washer. The apparatus works with the appliance control panel through wiring, radio waves, or other wireless methods and the control panel is programed to use the special water in the last rinse.

What is claimed is:

1. A method for getting dishes cleaner by using an apparatus called Lightning Clean Technology™ connecting to a dishwasher with a leak proof tank as a means to hold special water including the programer programed to run water into the leak proof tank at the beginning of the wash cycle and to run this special water onto the items at the last rinse.
